(19) United States
(12) Patent Application Publication
(10) Pub. No.: US 2023/0042585 A1
(43) Pub. Date: Feb. 9, 2023

(54) MULTI AXIS CORRECTIVE KNEE BRACE

(71) Applicants: Neil Naik, North West Calgary (CA); Vijaykumar Ganpat Naik, Pune (IN)

(72) Inventors: Neil Naik, North West Calgary (CA); Vijaykumar Ganpat Naik, Pune (IN)

(21) Appl. No.: 17/581,013

(22) Filed: Jan. 21, 2022

(30) Foreign Application Priority Data
Aug. 6, 2021 (IN) 202121035522

(51) Int. Cl. A61F 5/01

(52) U.S. Cl. CPC A61F 5/0123; A61F 2005/0148; A61F 2005/0137

(57) ABSTRACT

Disclosed is a multi-axis corrective knee brace. The knee brace comprises a thigh cuff component (101) configured to engage rear thigh area of an user, a calf cuff component (102) configured to engage with the patient's leg at the calf area below the knee, and a pair of multi-axial hinge assemblies (103) coupling the thigh cuff component (101) and the calf cuff component (102) for relative articulation. The knee brace further comprises rotating hinge elements (90) connecting the pair of multi-axial hinge assemblies (103) to the a calf cuff component (102) and to the thigh cuff component (101). Specifically, the rotating hinge elements (90) are capable of adjusted towards inner and outer side by means of key in order to treat other deformities such as Bow Legs (Genu Varum deformity) and Knock Knee (Genu Valgum deformity).

### CROSS-REFERENCE TO RELATED APPLICATIONS

The present application claims priority of Indian Application No.: 202121035522, filed on Aug. 6, 2021. The entire disclosure of the aforementioned application is incorporated by reference as part of the disclosure of the present application.

### FIELD OF THE INVENTION

The present invention generally relates to the field of osteoarthritis knee orthosis, more particularly, to a multi-axis corrective knee brace for osteoarthritis.

### BACKGROUND OF THE INVENTION

Osteoarthritis (OA) is a common form of arthritis. Primary osteoarthritis is typically related to the simple fact of aging and most often affects weight-bearing joints, such as the knee. This deformity is a complex condition that causes pain, stiffness, and limited movements of the knee joints. Osteoarthritis is caused when the Meniscus or cartilage of the knee joint gets damaged or worn out either due to an accident or an injury or overtime as natural aging progresses. The wearing out of the cartilage causes knee joints to narrow in distance, which results in knee joints rubbing against each other causing a decrease in its shock-absorbing capacity as well as a lot of pains and stiffness for the patient. Due to this narrowing of the distance, the normal walking alignment of the patient changes which results in limping. Repetitive use of a joint, such as the knee, over the years, is simply unavoidable and is wholly necessary for normal human function. It irritates and inflames the cartilage, thereby causing joint pain and swelling. If left untreated, this further causes other deformities such as Bow Legs (Genu Varum deformity), Knock Knee (Genu Valgum deformity).

Osteoarthritis is generally treated through an expensive Total Knee Replacement surgical procedure which cures the deformity through the use of implants.

However, the treatment creates other problems for the patient. The patient has to walk with the help of a tripod or a quadripod walking stick. The patient also cannot sit in a cross-legged or in a squatting position. Surgical treatment is required again as the lifespan of the implants is about 8-10 years. For some patients, there are some chances of the occurrence of infections, which again require treatment.

US Patent application US20100056970 discloses a knee orthosis device which provides medial, lateral, and rotational control of a knee joint. The novel knee orthosis device stabilizes an injured or surgically repaired, or a weakened knee joint having a neurological disability, which causes instability therein, by applying adjustable corrective and therapeutic forces, which operate to stabilize and protect the knee as well as to correct abnormal gait and to rehabilitate the knee joint and surrounding muscles above and below the knee by encouraging and recruiting said muscles to operate again more normally through the use of a dynamic swing assist mechanism. The knee orthosis also provides dynamically conformable thigh and shin cuff circumferential band systems in combination with a multitude of other elements that conform to the constant dimensional changes of the muscle groups that are surrounded by the knee orthosis device during ambulatory or athletic motion.

Another US Patent Application US2013004130 discloses a knee orthosis which has two vertical struts positioned on opposed sides of a knee joint in a generally parallel relationship. Each vertical strut has a hinge member located at a general middle portion; each vertical strut has a top and bottom end portion. An upper thigh cuff attaches at opposed ends to the upper end portions of the two vertical struts whereas a lower shin cuff attaches at opposed ends to the lower end portions. The lower shin cuff has a pair of hinges located proximal to the shin cuff opposed ends for permitting the shin cuff to pivot downwardly away from a shin of a person wearing the knee orthosis. An alternate embodiment employs hinges located proximal to the thigh cuff opposed ends for permitting the thigh cuff to pivot upwardly away from a thigh of a person when the same motion is performed.

However, both the US Patent Application disclosed above includes metal brackets which have to wear on front side of the lower leg having tibia bone. This may cause discomfort the user as the metal brackets may rub on the bones injuring them. Further, these patent application mentions use to vertical struts which does not allow flexibility of wearing as different user have different sizes.

Accordingly, there is a need for a multi-axis corrective knee brace to help in restoring the body's natural knee alignment.

### OBJECTS OF THE INVENTION

An object of the present invention is to provide a corrective knee brace for restoring the body's natural knee alignment.

Another object of the present invention is to provide off-loading braces to transfer the weight of the body from its anatomical knee joints to the brace's mechanical knee joints and uprights.

Still another object of the present invention is to cure osteoarthritis while completely avoiding a surgical treatment.

Yet another object of the present invention is to help patient with Osteoarthritis to stand and move around with more confidence and comfort.

### SUMMARY OF THE INVENTION

Accordingly, the present invention provides a multi-axis corrective knee brace. The knee brace comprises a thigh cuff component (101) configured to engage rear thigh area of an user, a calf cuff component (102) configured to engage with the patient's leg at the calf area below the knee, and a pair of multi-axial hinge assemblies (103) coupling the thigh cuff component (101) and the calf cuff component (102) for relative articulation.

The knee brace further comprises rotating hinge elements (90) connecting the pair of multi-axial hinge assemblies (103) to the a calf cuff component (102) and to the thigh cuff component (101). Specifically, the rotating hinge elements (90) are capable of adjusted towards inner and outer side by means of key in order to treat other deformities such as Bow Legs (Genu Varum deformity) and Knock Knee (Genu Valgum deformity).

### BRIEF DESCRIPTION OF THE DRAWINGS

The objects and advantages of the present invention will become apparent when the disclosure is read in conjunction with the following figures, wherein

FIG. 1 shows a perspective view of the multi-axis corrective knee brace, in accordance with the present invention;

FIG. 2 shows a side view of the multi-axis corrective knee brace, in accordance with the present invention;

FIG. 3 shows a bottom view of the multi-axis corrective knee brace, in accordance with the present invention;

FIG. 4 shows a top view of the multi-axis corrective knee brace, in accordance with the present invention;

FIG. 5 shows a close-up view of elements of the hinge assemblies of the multi-axis corrective knee brace, in accordance with the present invention;

FIG. 6 shows a close-up view of cuff brackets of the multi-axis corrective knee brace, in accordance with the present invention;

FIG. 7 shows a schematic drawing of the hinge mechanism for lateral turning of the knee brace in accordance with the present invention; and

FIG. 8 shows a schematic view of the knee brace when used by patient with genu varum deformity.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

The present invention provides a multi-axis corrective knee brace for osteoarthritis. The multi-axis corrective knee braces for osteoarthritis are known as off-loading braces. Further, the multi-axis corrective knee braces transfer the weight of the body from its anatomical knee joints to mechanical knee joints and “uprights” of the braces. The use of the corrective Knee Brace helps to restore the body's natural knee alignment if such a brace is used during weight-bearing activities like walking, climbing, etc. Additionally, this off-loading of the weight of the body causes the widening of the knee joints which results in the gradual restoration of the knee cartilage, curing osteoarthritis while completely avoiding surgical treatment.

The present invention is illustrated with reference to the accompanying drawings, throughout which reference numbers indicate corresponding parts in the various figures. These reference numbers are shown in the bracket in the following description.

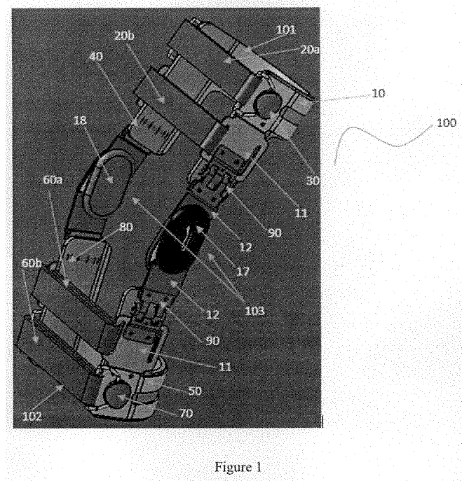

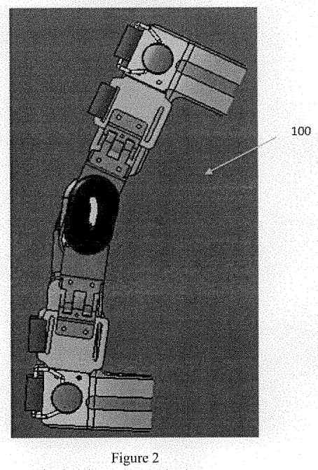

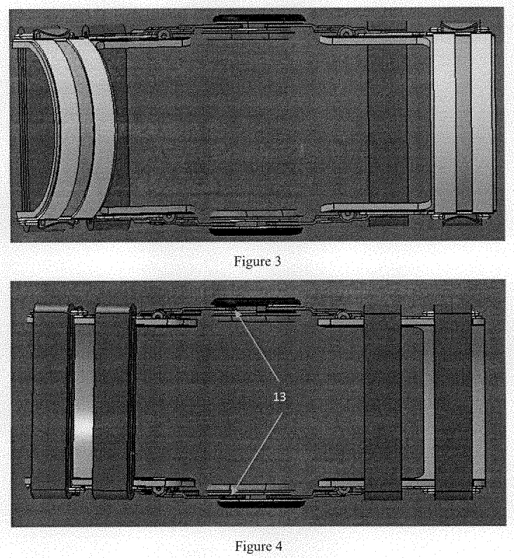

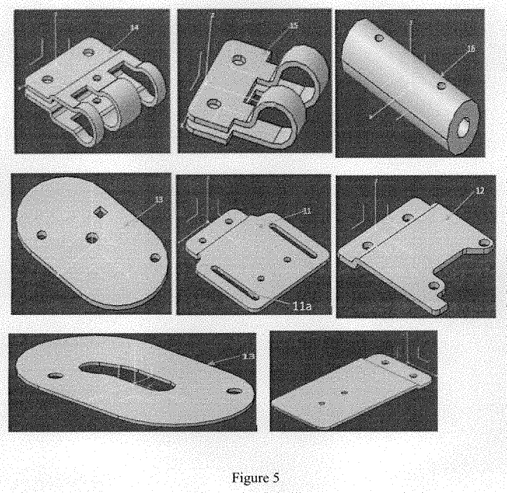

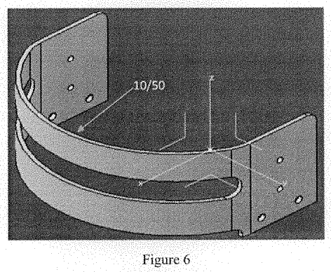

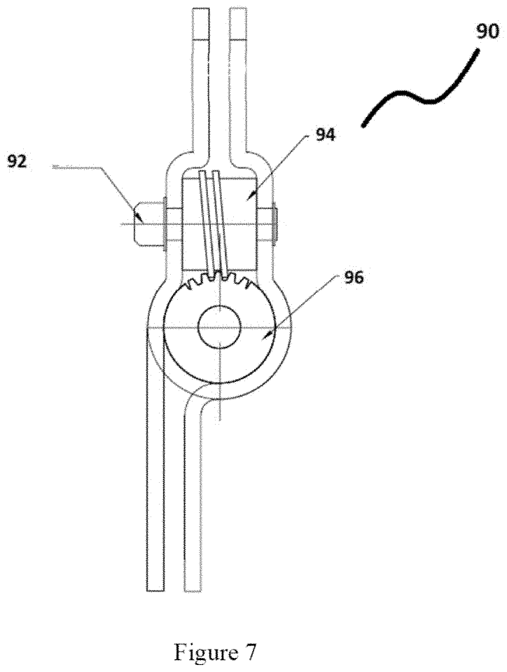

Referring to FIG. 1-8, there is shown a multi-axis corrective knee brace (hereinafter “the knee brace (100)”) to be used for treatment of bow legs (or genu varum) and the knock knee (Genu Valgum deformity). The bow legs (or genu varum) is the deformity when the legs curve outward at the knees while the feet and ankles touch. Whereas, the knock knee (Genu Valgum deformity) is a deformity in which the knees angle in and touch each other when the legs are straightened. Individuals with severe valgus deformities are typically unable to touch their feet together while simultaneously straightening the legs. The knee brace (100) of the present invention can be used to treat the above-mentioned deformities as the knee brace (100) can be adjusted towards inner outer side depending on the condition of the patent.

The knee brace (100) comprises a thigh cuff component (101), a calf cuff component (102), and a pair of hinge assemblies (103).

The thigh cuff component (101) engages with the patient's leg at the thigh area above the knee. Specifically, the thigh cuff component (101) engages from the rear of the thigh which provides flexibility to wearing. The thigh cuff component (101) comprises a thigh side bracket (10), a pair of thigh strap members (20a, 20b), an attachment clip (30), and a thigh side inner cushioning (40). The thigh side (10) bracket is made of sturdy metal such as steel, CRCA steel sheet similar metals. The thigh side bracket (10) is semicircular in shape and flexible enough the hold back thigh portion of the user.

The thigh side bracket (10) fits the exterior portion of the wearer's thigh. The thigh side bracket (10) is attached with the attachment clip (30) at both ends for attaching the thigh strap (20a). The pair of thigh strap members (20a, 20b) allows fitting of the thigh side bracket (10) with the thigh. In preferred embodiment, the pair of thigh strap members (20a, 20b) comes on rear portion of the thigh which provides ease to user. The pair of thigh strap members (20a, 20b) allows flexible fixation and gripping corresponding to the size of the wearer's leg being on front side. The thigh side inner cushioning (40) is located inside the thigh side bracket (10). The thigh side inner cushioning (40) provides support and comfort to the patient and restricts any discomfort that may occur due to friction of the thigh cuff component (10) thereon.

In an embodiment, the thigh straps (20a) are made of neoprene or similar material which is durable.

The calf cuff component (102) engages with the patient's leg at the calf area below the knee. This engagement of the calf cuff component (102) provides ease of the wearing to the user as against brace of the prior art which have to be wore from rear side of leg below the knee. The calf cuff component (101) comprises a calf side bracket (50), a pair of calf strap members (60a, 60b), an attachment clip (70), and a calf side inner cushioning (80). The calf side bracket (50) is semicircular metal component which fits the exterior portion of the wearer's calf. The calf side bracket (50) is attached with the attachment clip (70) at both ends for attaching a calf strap (60a). The pair of calf strap members (60a, 60b) allows flexible fixation and gripping corresponding to the size of the wearer's leg being on front size. The calf side inner cushioning (80) is located inside the calf side bracket (50). The calf side inner cushioning (80) provides support and comfort to the patient and restricts any discomfort that occurred due to the friction of the calf cuff component (102) thereon.

The pair of hinge assemblies (103) couples with the thigh cuff component (101) and the calf cuff component (102) for relative articulation. The hinge assemblies (103) are multiaxial hinges which allows easy adjustment irrespective size of knee of the user. The pair of hinge assemblies (103) is attached on both ends of the thigh side bracket (10) and the calf side bracket (50). Thus, the pair of hinge assemblies (103) provides support to the knee on both sides thereof. Further, the pair of hinge assemblies (103) comprises a pair of lateral hinge plates (11), a pair of rotating hinge elements (90), a pair of median links (12), and a pair of supporting hinge members (13).

The rotating hinge elements (90) connects the pair of multi-axial hinge assemblies (103) to the calf cuff component (102) and to the thigh cuff component (101) at all four locations ie. at lateral and medial portion in thigh location and lateral and medial portion of calf location. The rotating hinge elements (90) includes allen screw which can be adjusted to move the rotating hinge elements (90) towards inner side or outer side by means of allen key in order to treat other deformities such as Bow Legs (Genu Varum deformity) and Knock Knee (Genu Valgum deformity) as the case may be.

Specifically the allen screw of the rotating hinge elements (90) is rotated in clockwise direction to bring the rotating hinge elements (90) towards outer plane so that patient with Knock Knee (Genu Valgum deformity) can be treated. This provides firm support from inner side to the knee and helps pushing the knee to the outer plane.

Further, the allen screw of the rotating hinge elements (90) is rotated in anti-clockwise direction to bring the rotating hinge elements (90) towards inner plane so that patient with Bow Legs (Genu Varum deformity) can be treated. This provides firm support from outer side to the knee and helps pushing the knee to the inner plane.

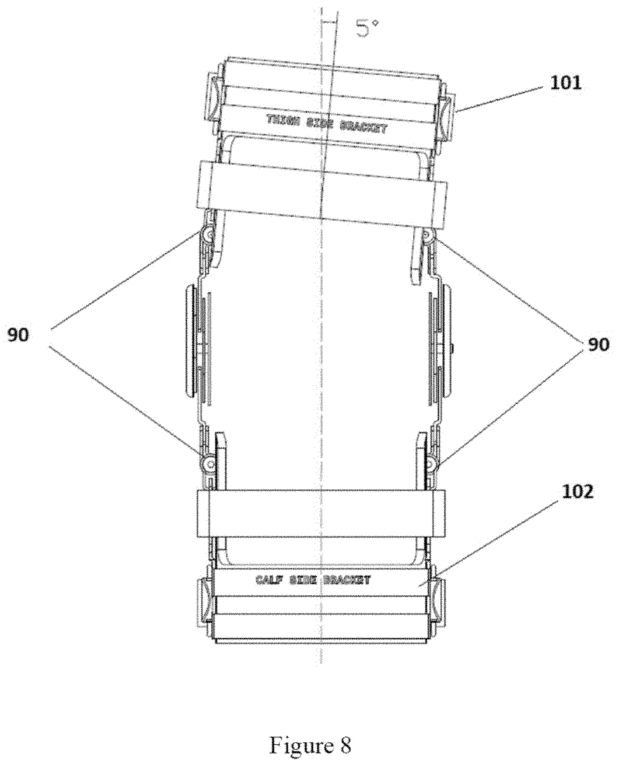

Specifically, FIG. 7 shows a schematic drawing of the hinge elements (90). The hinge elements (90) includes an adjustable allen screw (92) operably attached to the worm gear (94) which inturn is coupled with worm wheel (96) having toothed projection. When the adjusting screw (92) rotated is clockwise direction, the hinge mechanism (90) knee brace (100) moves laterally as shown in in FIG. 8.

The pair of the lateral hinge plates (11) are provided on both sides of the knees. Each hinge plate of the pair of the lateral hinge plates (11) couples with the thigh side bracket (10) and the calf side bracket (50) respectively. Further, each hinge plate from the pair of the lateral hinge plates (11) includes a pair of slots (11a) at both sides. The slots (11a) are provided for attaching the strap members (20b, 60b) for providing additional gripping of the knee brace (100).

The pair of rotating hinge elements (90) allows sideway rotation thereto. The pair rotating hinge elements (90) comprises a pair of two-point hinge elements (14), a pair of three-point hinge elements (15), and a pair of hinge pin (16).

Each element from the pair of three-point hinge elements (15), at one end thereof, couples with the lateral hinge plates (11) of the thigh side bracket (10) and the calf side bracket (50) respectively on upper and lower sides of the knee brace (100). The two-point hinge elements (14) engage with the three-point hinge elements (15) and locks therein by means of the hinge pin (16).

The pair of two-point hinge elements (14), on the other end, couples with the pair of median links (12). The pair of the median links (12) connects by pair of supporting hinge members (13) resulting in the making of a hinge point at the knee.

The pair of supporting hinge members (13) are covered by a cover (17) on the exterior side thereof. Further, the pair of supporting hinge members (13) are provided with a knee side cushioning (18) to provide support and avoid any discomfort therein.

### ADVANTAGES OF THE INVENTION

1. The knee brace (100) is very simple in construction and provides natural knee alignment while walking, climbing, etc.

2. The knee brace (100) gradually restores the knee cartilage.

3. The knee brace (100) cures osteoarthritis, by completely avoiding a surgical treatment.

4. The knee brace (100) helps one to stand and move around with more confidence and comfort.

5. The knee brace (100) has a low maintenance as compared to other available braces.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the claims of the present invention.

What is claimed is:

1. A multi-axis corrective knee brace for osteoarthritis comprising
a thigh cuff component (101) configured to engage rear thigh area of an user;
a calf cuff component (102) configured to engage with the patient's leg at the calf area below the knee,
a pair of multi-axial hinge assemblies (103) coupling the thigh cuff component (101) and the calf cuff component (102) for relative articulation; and
a rotating hinge elements (90) connecting the pair of multi-axial hinge assemblies (103) to the a calf cuff component (102) and to the thigh cuff component (101), the rotating hinge elements (90) being capable of adjusted towards inner and outer side by means of key in order to treat other deformities such as Bow Legs (Genu Varum deformity) and Knock Knee (Genu Valgum deformity).

2. The multi-axis corrective knee brace as claimed in claim 1, wherein rotating hinge elements (90) comprises a adjustable allen screw (92) operably attached to the worm gear (94) which in turn is coupled with worm wheel (96) having toothed projection.

3. The multi-axis corrective knee brace as claimed in claim 1, wherein the pair of hinge assemblies (103) is attached on both ends of the thigh side bracket (10) and the calf side bracket (50) for providing support to the knee on both sides thereof.

4. The multi-axis corrective knee brace, wherein the thigh cuff component (101) comprises:
a thigh side bracket (10) fits the rear exterior portion of the wearer's thigh, the thigh side bracket (10) is attached with the attachment clip (30) at both ends for attaching the thigh strap (20a).
a pair of thigh strap members (20a, 20b) allows fitting of the thigh side bracket (10) with the thigh. The pair of thigh strap members (20a, 20b) allows flexible fixation and gripping corresponding to the size of the wearer's leg.
an attachment clip (30), and
a thigh side inner cushioning (40) for providing support and comfort to the patient and restricts any discomfort that may occur due to friction of the thigh cuff component (10) thereon.

5. The multi-axis corrective knee brace, wherein the calf cuff component (101) comprises a calf side bracket (50), a pair of calf strap members (60a, 60b), an attachment clip (70), and a calf side inner cushioning (80).
